(19) United States
(12) Patent Application Publication
(10) Pub. No.: US 2025/0189388 A1
(43) Pub. Date: Jun. 12, 2025

(54) STRETCHABLE OPTICAL FIBERS FOR STRAIN-SENSITIVE TEXTILES

(71) Applicants: UNIVERSITY OF LOUISVILLE RESEARCH FOUNDATION, INC., Louisville, KY (US); CORNELL UNIVERSITY, Ithaca, NY (US)

(72) Inventors: Cindy HARNETT, Louisville, KY (US); Huichan ZHAO, Ithaca, NY (US); Robert SHEPHERD, Ithaca, NY (US)

(21) Appl. No.: 18/771,692

(22) Filed: Jul. 12, 2024

Related U.S. Application Data
(63) Continuation of application No. 16/965,444, filed on Jul. 28, 2020, now abandoned.
(60) Provisional application No. 62/623,078, filed on Jan. 29, 2018.

(51) Int. Cl. G01L 1/24; A61B 5/00; A61B 5/11; G01B 11/16; G02B 6/02

(52) U.S. Cl. CPC G01L 1/242; G02B 6/02; A61B 5/1107; A61B 5/6833; A61B 2503/10; A61B 2562/0266; G01B 11/18

(57) ABSTRACT

Optical fibers, optical waveguides, optical sensors, and combinations thereof are disclosed. Strains in excess of 100% are permitted while subcentimeter changes in fiber length are detectable. Light intensity changes with changes in fiber or waveguide strain, and the changes are correlatable with other variables. Production of such devices according to some embodiments entails coating of a core with cladding. Core material may be polyurethane whilst cladding material may be silicone, for example.

### CROSS-REFERENCE TO RELATED APPLICATIONS

This application claims the benefit of U.S. provisional patent application No. 62/623,078 filed Jan. 29, 2018, the complete contents of which are incorporated herein by reference.

### FIELD OF INVENTION

The pertinent field involves intrinsically stretchable fibers that serve as optical waveguides transmitting light for use in systems and methods that measure and characterize fiber motion under strain, for example when worn by a person during physical activity. Among multiple embodiments described herein, inventive fibers can be attached to textiles using a sewing machine and prestrained for monotonic intensity-vs-strain behavior. Installed in a stretchable piece of athletic tape or the like, they detect strains exerted by muscles during weight bearing.

### BACKGROUND

Although existing wearable sensors can detect the wearer's motion frequency and sometimes absolute position, a common difficulty is determining the user's exertion level without adhering electrodes to the skin to measure muscle signals. For example, resistive electronic signals need a continuous conductive path, which can be challenging to maintain between a dynamic soft actuator and a rigid detection circuit. Some of the most widely used methods to embed inextensible glass fiber optics, conductive fibers and films, semiconductor materials, and other integrated sensors in stretchable substrates involve buckling, coiling, knitting, sewing, and photolithography. However, it is a challenge to maintain a continuous path required for certain types of sensing under these conditions.

To overcome various challenges associated with electrical signals, optical stretch-and-bend sensors may be used which are capable of crossing an air gap, thus requiring less direct contact with a subject to transmit signals about the subject. Polydimethylsiloxane (PDMS) silicone elastomer demonstrates a high visible light transmission and elastic deformability. Accordingly, waveguides have been made from PDMS that utilize either a reflective metal coating or total internal reflection at the PDMS-air interface to transmit light and provide a stretchable optical communication link.

In addition, optical fiber sensors have included fibers embedded in soft silicone and incorporated with fabrics. In terms of practical application, optical fiber technology involving non-stretchable plastic components has been used to measure a person's breathing rate based on variations in light intensity due to longitudinal gap changes between two separated fibers, which are created by movement of the fiber during inhale and exhale processes, as part of patient monitoring.

Besides those mentioned above, a number of materials have been explored for optical fiber technology, including polyurethane fibers as wave guides for the transmission of light. However, bare polyurethane fibers show a degree of optical signal loss that is not conducive to accurate sensing, and is compounded when light must travel around bends having a radius of less than 1 cm.

Myriad additional applications exist and will be easily discerned for which the ability to interpret the intensity signal from textile-embedded stretchable fibers is needed, where the fibers must interact with the textile itself, the overlying threads, and any surfaces underneath. Unfortunately, however, cost and material incompatibility prevent the large scale integration of optical sensory fibers into clothing. Improvements in materials and methods will increase the practical use of expandable optical fiber sensing.

### SUMMARY

Disclosed herein are soft, stretchable, sewable and strain-sensitive optical fibers that provide intrinsically stretchable optical waveguides capable of >100% strain. In some embodiments, these are formed by coating a commercially available core fiber (e.g., of polyurethane) with a cladding (e.g., of silicone) having a lower refractive index than the core material. These fibers are then attached to textiles using a sewing machine which may be a conventional sewing machine.

According to an aspect of some exemplary embodiments, claddings are applied to a core fiber by one or more coating techniques and not by co-extrusion with the core fiber. A variety of advantages exist for coatings over co-extruded claddings. First, a wider variety of materials are available, as discussed in greater detail below. Second, coating equipment is generally less expensive than extrusion equipment. Third, coating permits manufacturers to customize small batches of extruded materials. For instance, a large amount of core fiber may be produced by extrusion and then subdivided into multiple small batches subject to different types or numbers of coatings. Fourth, coating equipment can be chained to apply double coatings (e.g. triaxial core-cladding-jacket structures) or more complex structures. Fifth, liquid-cure coatings may have additives mixed into them including but not limited to particles, dyes, and biological material (e.g., bacteria or biofilm). Some of these additives are not compatible with extrusion methods. For those additives that may be extruded as a matter of material properties, uniform mixing may require melting polymer pellets, mixing to achieve blending, and re-pelleting (requiring specialized machinery). High melting temperatures (e.g., 180 to 220° C.) required for some extrusion processes may also damage desired biological additives compared to coating methods, some of which may be performed at relatively low temperatures or even room temperature.

A wider variety of materials are available for coating compared to co-extrusion in part because co-extrusion requires core and cladding materials to be extruded in the same temperature and pressure range. Extrusion of a thermoplastic polymer onto a previously-extruded core can be considered a subset of coating; this extrusion coating method is used to apply insulating coatings to wires in the wire and cable industry, for example. Coatings may use or consist of photocurable substances, thermally cured materials, suspensions, solvent-based systems, and/or two part mixtures. These materials include substances that do not melt and cannot be processed using extrusion.

Some exemplary coatings example are stretchable materials with high optical transmission (for claddings). For jackets, the materials still need to be stretchable but may be opaque.

One example of a coatable but not extrudable material is a liquid-cure elastomer. To create solid coatings, these materials are coated onto existing fibers (e.g., an extruded core) as liquids, then permanently cross-linked into solids using ultraviolet (UV) light, a chemical reaction in a two-part mixture, or heating. Because the crosslinks are irreversible, they cannot be re-melted. Heating and UV curing (with an added photosensitizer) speed up the solidification of most of these cross-linkable liquids, so a single material may have several different processing methods described in the technical literature available to those of skill in the art.

Coating is also advantageous to address issues of nonconcentricity of fibers layers and exposed cores. With these fibers, perfect concentricity is not needed, but a thin spot in the cladding (e.g., less than ˜5 micron) is a source for a light leak. If an extruded fiber is found to have an off-center or exposed core, room-temperature coating can be applied to bulk up the cladding without melting the existing structure.

Once manufactured, exemplary fibers according to some embodiments may be installed in a textile such as but not limited to a stretchable piece of athletic tape or flexible elastic compression bandage to detect strains originating from changing muscle shapes during weight-bearing activity.

In some embodiments, an optical fiber comprises a stretchable core material having a first end and a second end; and a cladding surrounding the stretchable core material for reducing optical signal loss, wherein the cladding has a lower refractive index than the stretchable core material, and wherein the optical fiber is capable of transmitting an optical signal comprising light waves traveling from the first end to the second end.

Some embodiments are strain sensors which may include an emitter for injecting light into the first end of an optical fiber and a receiver to detect the light which reaches the second end of the optical fiber. The emitter may be, for example, an LED or LED array. The receiver may be a photodector such as a photodiode or photodiode array.

Fibers according to some embodiments may be attached to textiles using, e.g., a sewing machine. Fibers according to some embodiments may be prestrained for monotonic intensity-vs-strain behavior. Prestraining compensates for microbends in the fiber which are sometimes created by threads which attach the fiber to a textile.

When installed in a stretchable piece of athletic tape or other material which is configured for wrapping snugly about a body part, fibers may detect strains exerted by muscles during weight bearing. In some embodiments, one or a plurality of such optical fibers are used in a sensing system which also comprises a detector (e.g., photodetector) positioned proximal to the second end, wherein the detector is in communication with a processor for processing the signal associated with transmitted light.

Optical fibers configured as strain sensors bear the advantages of not having to use metal or conductive particle fillers and not being burdened by the need for direct contact at all points of transmission. In short, though optical fibers according to some embodiments may be nonconductive (e.g., consisting of insulative materials), they may provide detection functionality previously reserved for electrically conductive thread while avoiding many of the restrictions and drawbacks of electrically conductive thread.

Other advantages achieved with present embodiments include that fiber optics are generally lighter weight and lower cost than conductive polymer composites, metals, or silver-plated conductive yarns. Optical paths are not broken by oxidation or corrosion in wet environments, a common problem with conductive paths when a textile with metallized yarns is washed. Their high light transmission means light can get past sensors to a solar array, or the fibers themselves can pick up ambient light and channel it to a solar cell. Moreover, with sewn on optical fibers, the sensors of present embodiments can add sensing capability to a garment even after construction, in contrast to weaving, knitting, or ink-based printing methods, which can have difficulty crossing seams.

Biomedical applications for optical fibers of some present embodiments can be achieved by integrating them into soft, stretchable fabrics and materials. Such applications include placing a wearable strain sensor on a piece of athletic tape, such as by stitching. It has been determined that stitching a zig zag stitch over the top of fibers disclosed herein will hold it in place during stretching of the underlying textile. An inexpensive couching machine foot may be used to create uniform stitches without damaging the fiber. Exemplary optical fibers disclosed herein are compatible with a basic sewing machine. Optical fibers according to the invention could be installed in a blanket, sheet or other covering and placed on a resting patient in a hospital to monitor respirations based on the frequency and intensity of rising and lowering chest movements as indicated by the transmission of light through the optical fibers.

Other applications of sensors according to some embodiments are body-scale sensors (e.g. 10-50 cm in length). In some applications, red LED light is capable of being detected easily through about 50 cm of fiber. Moreover, with a lossier wavelength (550 or 915 nm), the sensitivity may be adjusted upward for sub-10 cm sensors (e.g, 1 to 10 cm length). Additional examples of potential uses involve computer-controlled couching for creating structures with deliberate stress concentration, to amplify strain signals or focus measurements on a specific region.

With improved materials and configurations according to some embodiments disclosed herein, it is possible to more effectively and reliably guide light through fiber optic sensors in wearable, stretchable fabrics. Many applications become available. Examples include stretchable optical communication networks, motion/bending sensors for prosthetics, fluidically powered soft actuators, sensor-embedded garments, and wraps or bandages aware of a user's position. Available uses further encompass sensory fibers in contact with the human body to generate population-scale insights into disease spread, gait analysis, and short- and long-term posture trends which are relevant to conditions ranging from traumatic injury to the progression of osteoporosis. Moreover, optics-based systems can be used in MRI chambers and other locations with high electromagnetic interference.

Some embodiments include a low-cost, light-guiding thread for sewing advanced and durable textiles capable of, for example, proprioceptive and exteroceptive sensing. The stretchable optical fiber produced by the current teachings herein can be attached to textiles using a sewing machine, wherein the optical signal is transmitted as a function of mechanical strain up to and in some cases exceeding 100%. In addition to their sensing capabilities, as manufactured the stretchable optical fibers of some present embodiments handle like threads to add high strain capability to the textile into which they are sewn or otherwise incorporated.

Many other features and advantages may be realized through the practice of the present embodiments, as explained in further detail below.

### BRIEF DESCRIPTION OF THE DRAWINGS

FIGS. 1A and 1B illustrate an optical fiber according to multiple embodiments and alternatives, comprising a polyurethane core with silicone cladding for the transmission of light at wavelengths above about 600 nm with peak in near infrared part of spectrum.

FIG. 1C illustrates a strain measurement system involving optical fibers.

FIGS. 1D and 1E illustrate unstrained and strained fibers, respectively, arranged for transmission of light from a light emitting diode (LED).

FIG. 2A graphs intensity loss versus strain associated with stretching for three different optical fibers.

FIGS. 2B-2E graph further optical and mechanical properties of silicone-clad polyurethane optical fibers according to multiple embodiments and alternatives.

FIGS. 3A and 3B illustrate systems for coating a fiber with one or more claddings for improved light transmission.

FIGS. 4A and 4B provide illustrations of a couching arrangement to align fiber inside a sewn zig-zag stitch, and of how stretching of the textile controls the optical transmission as a couch fiber undergoes deformation through stretching.

FIG. 4C illustrates a strain measurement system for optical fibers integrated with a textile or like device such as athletic tape.

FIG. 4D shows behavior of a couched fiber.

FIG. 4E is a plot that shows five cycles of stretching and relaxing the tape with embedded fiber.

FIG. 5A depicts cross-sectional changes and lengths associated with non-weight bearing and weight bearing activity when a sensor is worn on the back of the knee. FIG. 4A was obtained using a Microsoft Kinect to capture scans of weight bearing and non-weight bearing knees. Cross-sections were extracted from the Kinect data.

FIG. 5B graphs sensor output vs time during ten weight-bearing cycles in accordance with FIGS. 4A-B. The data in FIG. 5B were captured over ˜1 m long wires to the data collection system. Length changes in the fiber were measured with a measuring tape and were in the same range as measured by the 3D scanner in FIG. 4A.

FIG. 6 provides an exemplary detection circuit for transmitted optical signals obtained from the optical fibers of present exemplary embodiments.

### DETAILED DESCRIPTION

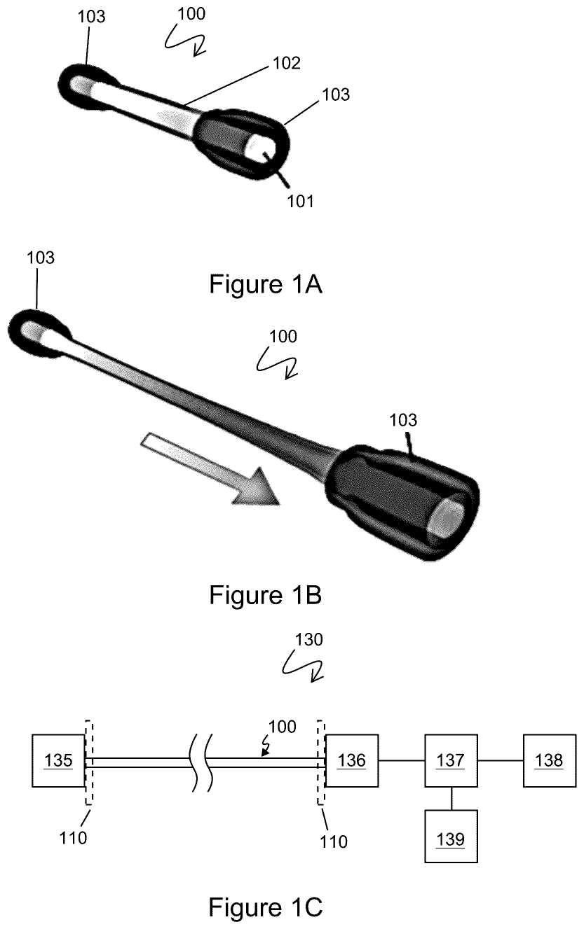

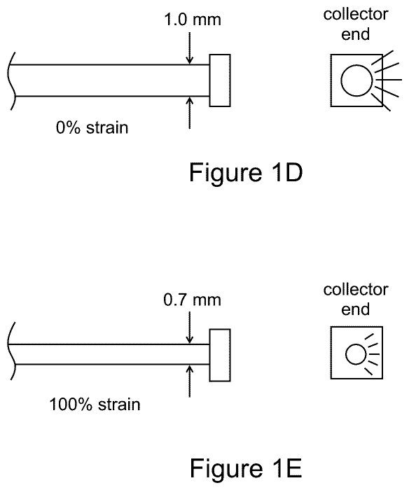

FIG. 1A is an exemplary light-guiding device 100. For convenience and consistency of description, the device 100 may be referred to hereafter as a light-guiding thread 100 or simply thread for short. Exemplary devices may also be referred to as “optical fiber” or simply “fiber” depending on the context. Devices like device 100 according to this and other exemplary embodiments may be characterized as, for example, optical waveguides, optical fibers, fibers, ribbons, strips, threads, strings, yarns, filaments, strands, and chords.

A thread 100 may be incorporated into, for example, advanced textiles capable of sensing one or more conditions. The thread 100 is stretchable. FIG. 1A depicts the thread 100 in a relaxed, unstrained state. FIG. 1B depicts the thread 100 in a stretched, strained state. Housing 103 may be provided at the first, second, or both ends of the thread 100.

Optical fibers and threads made therefrom generally have at least a core, a cladding, and in some but not necessarily all instances, a jacket. Multiple claddings may also be used in some cases. An exemplary thread 100 comprises a core 101 and at least one cladding 102 (e.g., one or more claddings). The core 101 has a higher index of refraction than the cladding 102. If there are multiple claddings, than the core has a refractive index greater than that of the coating which contacts the core. The index of refraction of the core may be required to be at least 0.05 greater than the index of refraction of the nearest cladding. The greater the index mismatch, the easier it becomes to couple light into the thread. As a result, with a larger mismatch, it becomes possible to send light over a wider cone of angles with respect to the fiber/thread axis.

The core 101 may consist of or comprise, for example, urethane. As used herein, “urethane” and “polyurethane” are interchangeable, with both equally referring to polyurethane. Besides urethane, other exemplary materials for the core 101 include higher refractive index (n at least 0.05 greater than the cladding), high transparency silicone elastomers, acrylic elastomers, and polyethylene elastomers.

The cladding 102 may consist of or comprise, for example, silicone. Besides silicone, other exemplary materials for the cladding 102 which may be used alone or in combination for various embodiments include but are not limited to urethane elastomers, acrylic elastomers, and polyethylene elastomers. These materials may be low refractive index (n<1.45) high transparency or semi-transparent. Among its functions, the cladding protects the core-cladding interface from dust and oils that cause light to scatter out.

Exemplary embodiments provide one or more (e.g., at least one) cladding in the form of a coating (or multiple coatings). Coatings may be or include one or more of biological materials (e.g., bacteria, biofilms, enzymes), photocurable substances, thermally cured/curable materials, suspensions, solvent-based systems, and two-part mixtures. Other biomolecular coatings which may be employed in some embodiments may be found in Leung, Angela, P. Mohana Shankar, and Raj Mutharasan. “A review of fiber-optic biosensors.” Sensors and Actuators B: Chemical 125, no. 2 (2007): 688-703, incorporated herein by reference. An exemplary coating may be a liquid-cure elastomer, including but not limited to a silicone liquid-cure elastomer, a urethane liquid-cure elastomer, and a fluorinated elastomer. Another alternative may be styrene. Such liquid-cure elastomers may be irreversibly cross-linked with one or more of ultraviolet light, a chemical reaction (as in a two-part mixture), and/or heat. Ambient temperature and/or humidity may also trigger or facilitate cross-linking.

To be useful as cladding material in stretchable optical fiber sensors in the ˜ 10 cm length range, the elastomer must have low optical transmission loss (loss of <10 dB/cm). The following description identifies a non-exhaustive list of some materials that may be used as claddings in some exemplary embodiments. Examples of UV curable liquid elastomers suitable for claddings: Siloprene liquid silicones (such as available at this address https://momentive.com/en-US/categories/elastomers/uv-cure-silicone-rubber/#), and Novagard UV curable silicones (such as available at this address https://www.novagard.com/novagard/uv-curable-sealants/) for claddings. Siloprene LSR-7000 specifically is designed for high optical transmission. Examples of highly stretchable (400-1000% elongation at break) two-part curable silicone elastomers suitable for claddings include EcoFlex 00-10, EcoFlex 00-30, and Dragon Skin from Smooth-On Inc., and M-4641 from Wacker Chemie. Clear, elastomeric, thermally cured silicones also include one-part RTV (rapid thermal vulcanization) silicones that react with air humidity to cure, for example Dow Corning RTV-3145. Besides silicones, urethane two-part curable elastomers are also available in clear formulations for claddings. For example, Clear-Flex from Smooth-On (such as available at this address https://www.smooth-on.com/product-line/clear-flex/). Fluorinated elastomer coatings are available as a suspension of nanoparticles as AFLAS-150cs. (such as available at this address https://www.agcchem.com/documentation/technical-data/aflas-fluoroelastomers/51-aflas-150cs-latex-product-information/file). These liquids are coated on surfaces and then air- or heat-dried for the solvent (water) to be removed. Examples of UV curable liquid elastomers suitable for jackets: ISOLENE liquid synthetic rubber (such as available at this address http://www.royalelastomers.com/brand.asp?division_id=2). It is opaque, making it more useful for jackets than claddings which are not jackets.

The core 101 may have, for example, a refractive index (n_{core}) of 1.52. The cladding 102 may have, for example, a refractive index (n_{cladding}) of 1.43. Generally, for some exemplary embodiments the core 101 has a refractive index at least 0.05 greater than that of the cladding 102. The above silicone materials have refractive indices in the range of 1.4-1.5, while urethanes are in the 1.5-1.6 range. Fluorinated elastomers meanwhile are in the 1.2-1.4 range. A reason to use fluorinated elastomers as claddings is their low refractive index compared to all other dielectric materials.

Together with the materials and their arrangement, dimensional characteristics of a thread 100 are configured to permit considerable flexibility of a thread 100. Advantages of flexible threads 100 include sewability, the ability to sew them into fabrics and textiles by conventional means such as but not limited to a sewing machine (e.g., a Brother PE770 or Tajima TLMX, for example). A core 101 may have a diameter between about 500 and 800 microns for example. A cladding 102 may have a thickness of 100 to 200 microns, for example. In some embodiments, the complete fibers/threads (including core, cladding(s), and jacket if applicable) range in diameter from 0.5 to 3 mm, or 1-2 mm, for example. Prototypical embodiments included urethane-core fibers with a cladding of 2-part thermally cured silicone where the difference of refractive index was 0.01 to 0.1, core diameter was 0.5-1 mm, and cladding thickness 0.2 mm to 1 mm. Further details of prototypical embodiments are discussed in the Example below.

FIG. 1C schematically shows a strain sensor 130 which comprises a thread 100 with an emitter 135 at one end and a detector 136 at the other end. The length of the thread 100 may be virtually any length suitable for incorporation into a textile. Some non-limiting example lengths are greater than 1 cm, greater than 2 cm, greater than 3 cm, greater than 4 cm, greater than 5 cm, greater than 6 cm, greater than 7 cm, greater than 8 cm, greater than 9 cm, greater than 10 cm, greater than 25 cm, greater than 50 cm, greater than 75 cm, greater than 100 cm, greater than 250 cm, greater than 500 cm. An upper limit, while not necessary in many embodiments, may be 100 cm, 500 cm, or 1000 cm, for example. Many though not necessarily all strain sensors for wearable applications may be configured in the 1 to 20 cm range, or 5 to 15 cm range, or 5 to 10 cm range.

The emitter 135 may consist of or comprise a light source such as one or more light emitting diodes (LEDs). The detector 136 may consist of or comprise a transducer for converting a received optical signal to a digital voltage signal. The transducer may further include, or be in a signal pathway/connection with, a processor 137 (or processors) configured to convert the digital (voltage) signal to a strain reading. The transducer may be, for example, a photodetector (e.g., photodiode) configured for measuring light transmission. The strain reading may then be transmitted by wired or wireless connection to an output device 138 which either displays or transmits the strain reading to a user or to a downstream device (e.g., a local or remote server or computer, a printer, a non-transitory storage console or medium like a solid state drive, etc.). One or more (e.g., all) elements downstream of the detector 136 be arranged as part of or on the same article of manufacture as the thread 100 or, alternatively, may be provided remote from the thread 100. Generally, the analog signal produced by a transducer (like a photodetector) may be amplified, filtered, and converted to a digital signal before recordation or output. Appropriate hardware that may be configured to perform these operations include but not limited to processor(s), microprocessor(s), specific purpose computer(s), general purpose computer(s), and supporting circuits, circuitry, and analog and/or digital circuit elements, and the like.

A thread 100 may be configured as an optical fiber which has an elastic response even when subjected to cycles of more than 100% strain followed by relaxation. The strain from which the thread 100 may routinely recover may be, for example, more than 100%, more than 110%, more than 120%, more than 130%, more than 140%, more than 150%, more than 200%, more than 250%, more than 300%. The allowable strain within which the thread may still exhibit a fully elastic response may be as great as 300%, 400%, or 500%, for example. Exemplary materials for cores and coatings (e.g., claddings and jackets) may be configured to withstand at least 400% elongation at break, e.g. 400 to 1000% elongation at break.

The thread 100 is configured to change optical transmission properties based on strain to which the thread 100 is subjected. For example, in some embodiments, light attenuation increases in a state of greater strain/stretch as compared to a state of less strain/stretch. The emitter 135 may be a white light source, for example. In such case, green and shorter wavelengths (e.g., 570 nm or less) may be filtered out through absorption when the thread 100 is stretched. The same wavelengths that are filtered out when the thread 100 is strained may be transmitted when the thread 100 is unstrained.

Unless indicated or implied otherwise, stretching or straining of a thread 100 refers to deformation along its primary (longitudinal center) axis. The detector 136 collects the fraction of the energy initially injected into the thread 100 by the emitter 135. The fraction of energy that reaches the detector 136 correlates with the strain of the thread 100 at the time of the emitter's energy transmission. The combinations of materials, dimensions, and configurations disclosed herein are advantageous in that they may permit detection of changes in light attenuation, and thus strain, on a miniscule scale (e.g., changes of 1 cm or even less than 1 cm). Generally, transmitted light intensity drops exponentially with length of a thread.

A processor 137 of a strain sensor may be configured to use known or preconfigured intensity-vs-strain tables or equations as a reference to translate real-time or substantially real-time intensity readings to strain readings. Such reference data or algorithms may be stored in a memory 139 accessible by or connected with processor 137.

FIGS. 1D and 1E depict reversible changes in physical characteristics of a thread 100 as well as transmission characteristics with respect thereto. FIG. 1D, for non-limiting illustrative purposes, depicts a thread with 0% strain and a total diameter of 1.0 mm. The intensity of light transmitted from emitter to detector (i.e., without being absorbed) is characterized in the figures using small lines that allow for qualitative comparison. Longer lines adjacent the end of the thread indicate comparatively greater transmission, and smaller lines adjacent the end of the thread indicate comparatively lesser transmission. FIG. 1E shows the same thread as depicted by FIG. 1D and with the same amount of light energy introduced to the thread by an emitter (not shown). However FIG. 1D shows the thread subjected to 100% strain. The elongation of the thread has reduced the total diameter from 1.0 mm to 0.7 mm, and the total energy transmission of the thread (from emitter to detector) has been significantly reduced.

The emitter 135 may produce optical energy of any of a variety of wavelengths. In some exemplary embodiments infrared wavelengths are used (e.g., 935 nm). In some embodiments visible light wavelengths are used. In some embodiments additional or alternative wavelengths of the electromagnetic spectrum are used.

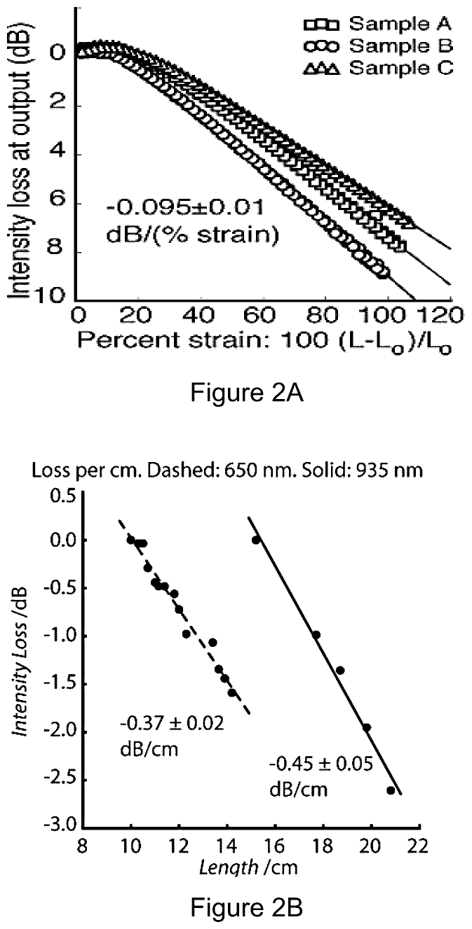

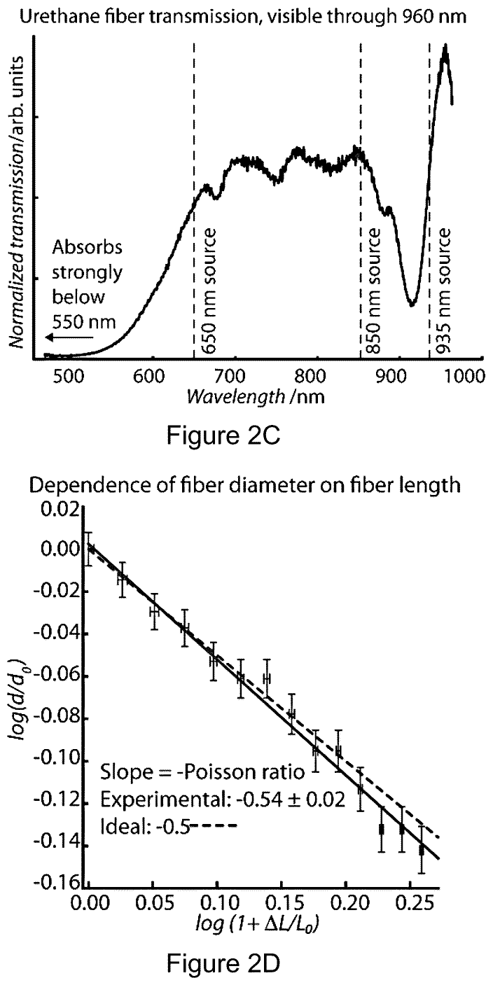

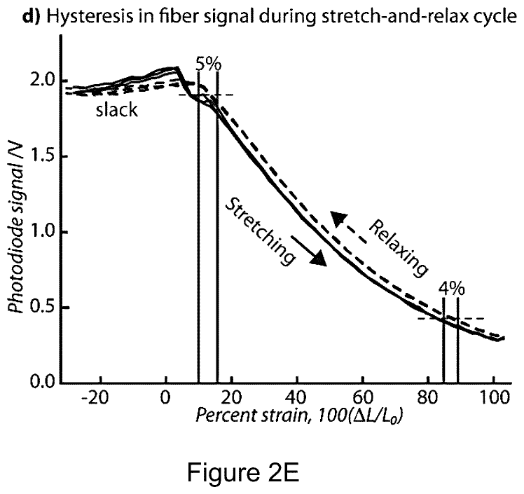

FIG. 2A presents by way of illustration intensity loss of sample embodiments as a function of strain. FIG. 2A is graphs of intensity loss vs strain for three different silicone-clad 0.5 mm diameter polyurethane fibers with a resting length of ˜21 cm; each trace contains data points from at least three successive relaxation cycles for an individual fiber. The transmitted light intensity drops exponentially with length, but with attenuation that can be detected on the cm scale instead of the kilometer length scale as seen in comparable systems such as silica fiber optics.

FIG. 2B presents illustrative absorption-vs-length for exemplary samples. The plot is illustrative of the fact that light is lost between the emitter and the detector from both absorbance and scattering combined. Additional light may be lost due to elimination of higher-order waveguide modes as the core diameter shrinks during stretching.

Some embodiments are configured such that the waveguide cord diameter (in a relaxed state) is much larger than the wavelength(s) of the light from the emitter 135. In this case the device 100 has N guided modes, where N may be determined by Equation (1):

$$N = {( \frac{4 a}{λ} )}^{2} ( {n}_{c o r e}^{2} - {n}_{c l a d d i n g}^{2} ) \qquad ( 1 )$$

where n_{core} is the index of refraction of the core, and n_{cladding} is the index of refraction of the cladding. As an example, with a=0.5 mm and λ=935 nm, there are ˜1.2×10⁶ guided modes. About half the modes disappear under 100% strain as the core contracts to 70% of its original diameter. Loss of high-order modes may not necessarily have a strong effect on transmitted power. Effects on transmitted power may be minimized in an embodiment through the emitter 135 being configured to launch low-order modes down the center of the fiber and by high-order modes scatting out quickly due to their high incidence angles with the cladding. The emitter configuration to launch low-order modes may be achieved with, for example, a plastic microlens so configured. As a result the high-order modes are likely to contribute minimal if not negligible power to the total power transmission in the original unstretched fiber. For extreme deformations during pinching and pressure, however, the changing shape of the core does cause a steep loss in transmitted intensity when light traveling parallel to the core encounters the distorted wall at an angle too steep for total internal reflection.

Changes in a fiber's total internal reflection angle may occur because of strain-induced changes in the materials' refractive index. However, it is known that mechanically tunable silicone elastomer diffraction gratings do not show strong variance of refractive index with strain. Furthermore, as with most elastomers, the fiber is nearly incompressible, so density-based refractive index changes are not expected.

FIGS. 2C-2E show other optical and mechanical properties of an exemplary silicone-clad/coated polyurethane fiber. FIG. 2C shows (normalized) transmission for wavelengths of approximately 500 up to 1000 nm. The polyurethane rubber transmits well at wavelengths between 650-1000 nm and possibly beyond, except for a low-transmission notch between 870-930 nm. White light transmitted through the polyurethane rubber core emerges as yellow, with approximately 20 times greater transmission intensity at 700 nm than 550 nm. The material is compatible with the visible red (650 nm) and near IR (850 nm) wavelengths used for optimum transmission in PMMA plastic optic fiber. Some specific wavelengths available with commercially available emitters are indicated in FIG. 2C using broken lines.

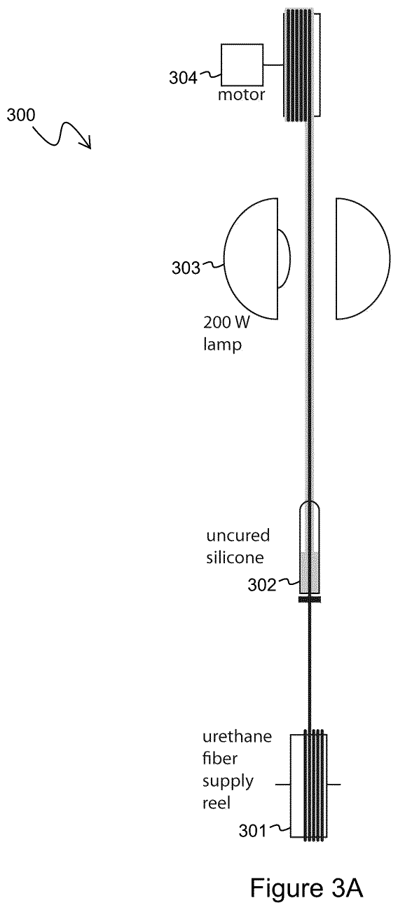

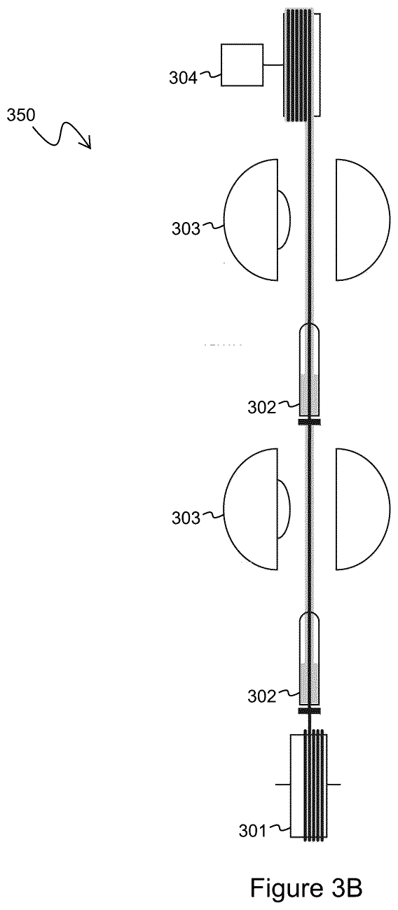

FIG. 3A shows a manufacturing apparatus 300 for producing an exemplary thread 100 in accordance with some embodiments. A real of urethane fiber supply 301 provides a continuous feed of the core fiber. The fiber from the real is passed through a vessel 302 of the cladding material, e.g. uncured silicone. At this stage the fiber is coated with the raw cladding material or materials, and then the coated fiber moves on to a curing stage. A silicone cladding coating may be cured with, for example, a heat lamp 303. After the cladding is cured the fiber may be complete and may be respooled by a motor 304. The fiber may be coated with a series of coatings, with curing steps between respective coating steps. The relatively few stages may advantageously allow the entire system to be driven by a single motor 304. For scaled production, additional motors and material pathways may be used.

FIG. 3B shows a manufacturing apparatus 350 adapted from apparatus 300 to illustrate application of multiple coatings (e.g., two or more) in a continuous manufacturing process. The manufacturing process may generally comprise extruding a center fiber from a first material (or obtaining pre-manufactured core from a known supplier), coating the center fiber with at least one other material after the center fiber is extruded, and curing (e.g., cross-linking) the at least one other material, such as with ultraviolet light, a chemical reaction as in a two-part mixture, and/or heat. A variety of coating techniques may be employed, including but not limited to dip coating (e.g., concentric dip coating). The multitude of coatings may be configured differently among different embodiments. For instance, in some embodiments, two adjacent coatings may be identical material and constitute a single cladding. In some embodiments one or more coatings may form one or more claddings, while a final coating constitutes a jacket.

A coating which is a jacket may be opaque to screen ambient light from reaching the extruded core. Prototypical signal processing of a light-guiding thread without an opaque jacket shows that some ambient light may enter the light-guiding thread through clear material. This is not unworkable, as the ambient light can be compensated for with a processor after making a measurement with the emitter (the intended light source injecting light into the fiber) on and another measurement with the emitter off. A difference of the two measurements may be taken and the result removed from future measures (assuming the ambient light is constant or near constant over a sufficient duration of time). An opaque jacket applied by coating has the advantage that processing steps to eliminate the effects of ambient interference may be avoided, and remaining data collection and processing may proceed that much faster.

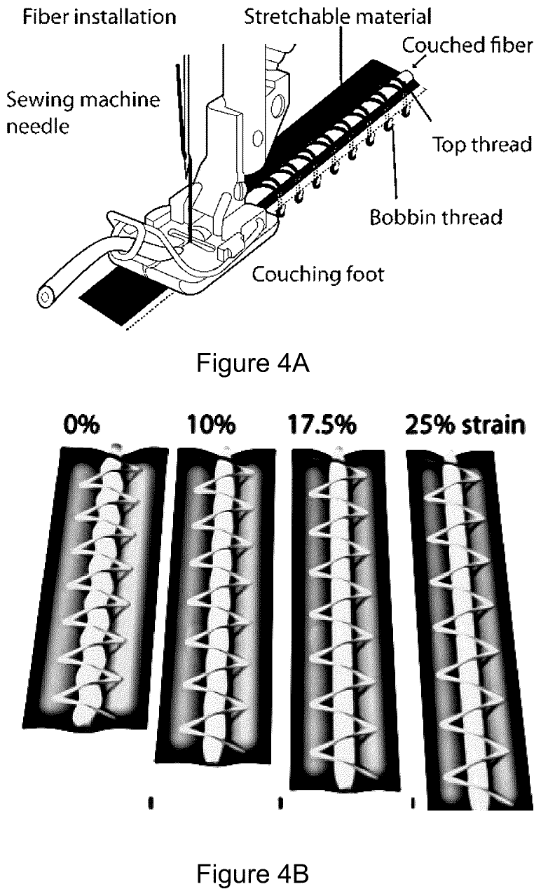

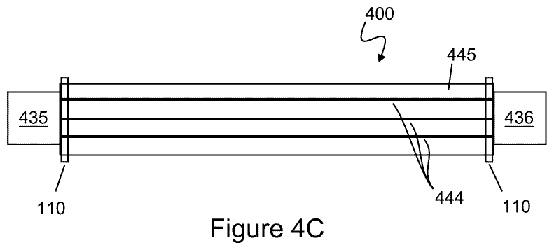

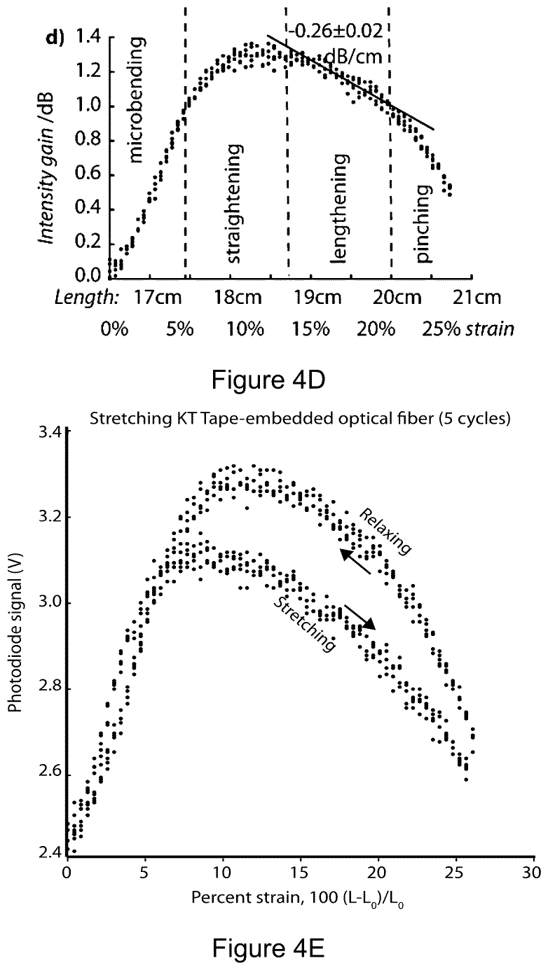

FIGS. 4A-4E illustrate fiber installation in a textile. In particular, FIGS. 4A and 4B depict couching and a couched fiber. In this case, couching uses a sewing machine foot to align a thick fiber inside a zig-zag stitch on a sewing machine.

A consequential aspect of some embodiments is sewability of a light-guiding thread. Yet, the sewing process may affect the signal transduction of the light-guiding thread and therefore the signals themselves. As depicted in FIGS. 4A and 4B, for example, light-guiding threads (optical fibers) may be attached to a textile by essentially “trapping” the light-guiding thread to the textile using another (non-light-guiding, say “plain”) thread made of conventional textile materials such as not limited to cotton, polyester, wool, nylon, or some combination of these. When the light-guiding threads are trapped under machine-sewn threads, the latter can actually change the cross-section and light transmission of the former. To account for deformations and alterations in physical shape and signal performance, embodiments herein may employ a number of solutions. One solution is the use of multi-layer claddings applied as coatings. For instance, some embodiments may comprise a double layer of cladding (e.g., silicone cladding) to reduce the amount of microbending caused by threads pressing into the soft light-guiding thread.

Another feature of some embodiments which improves sewability is prestrain. A strain sensor comprising a textile and one or more light guiding threads attached to the textile may provide, for example, prestraining of the threads by 15-25%. The prestrain may be supplied by threads of the textile, e.g., a backing. Prestraining (or prestretching) promotes monotonicity of the strain sensor response, in particular the intensity vs strain behavior. Pre-stretching the textile (e.g., tape or fabric) by about 15-25% helps achieve monotonicity by removing microbends in the structure. For stretching beyond 25% the light transmission may begin to decrease too dramatically as the optical path becomes longer.

Accordingly an exemplary method of using a wearable strain sensor that comprises a textile and one or more optical fibers attached to the textile and having elastic responses for strains of up to at least 100% may comprise steps of prestraining the textile and one or more optical fibers by 15-25% and fixing the wearable strain sensor to or about a subject (say a person, animal, or machine) such that the prestrain is maintained during a period of use, wherein the prestrain is configured to remove microbends in the one or more optical fibers to promote monotonicity of strain sensor response.

While extrusion is an exemplary process for producing a core (a core fiber), it is not the only means of doing so. Molding and variations on molding and/or extrusion, now known or developed in the future, may produce cores suitable for use with at least some embodiments of the invention. Embodiments herein which describe cores as being extruded may alternatively employ cores made by alternatives or variants to extrusion.

FIG. 4B shows that as the fiber stretches, microbending is reduced but the path length increases. These are competing effects and may result in transmitted light intensity increasing with strain until approximately 10-15% strain, where it begins a monotonic dropoff. As the fiber stretches, it affects the optical transmission of the deformable couched fiber even as they further interact with the surface and the threads.

FIG. 4C illustrates a strain measurement system 400 for optical fibers integrated with athletic tape. In FIG. 4C fibers 444 are installed in an athletic tape 445 installed with an LED light source 435 and light detector 436 positioned at opposite first and second ends of the fibers. FIG. 4C shows a setup similar to that which is depicted in FIG. 1C, modified to further comprise a textile to which the fibers have been attached or otherwise integrated. Couching is one of the means by which a fiber may be installed in or on a textile. Devices 110 which may be, for example, displaceable clamps, may be arranged for the purpose of systematically stretching threads, such as for testing purposes.

FIGS. 4D-4E illustrate couchings' effects on the signal transmitted through the fiber. Couching the fiber to a surface as depicted in FIG. 4A introduces compression and microbending in the fiber.

Fibers and textile with integrated fibers are useable in a wide variety of applications including but not limited to wearable sensors. Wearable sensors according to some embodiments may be configured for gait sensing, sports applications, and physical therapy, for example. A wearable sensor which may configured to include or consist of an embodiment herein may range from pedometer style step-counters to multi-axis accelerometers that measure joint angle.

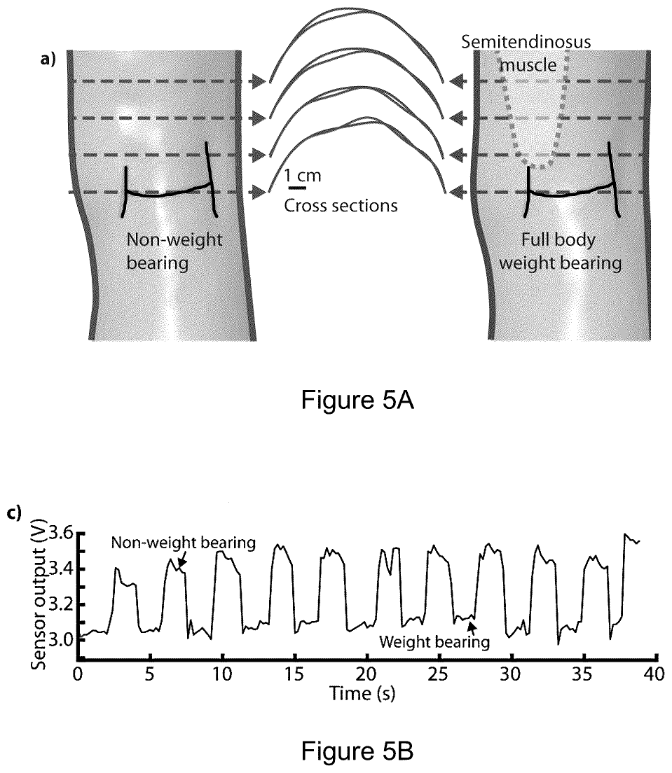

FIG. 5A illustrates a wearable sensor for joint monitoring. Stretchable athletic tape with integrated fibers is placeable above the knee, for example, to monitor muscle contract in the leg. Even small deformations, e.g., as small as 1 cm, are detectable and useable by the device processor to determine the leg is or is not under loading (weight bearing). It will be appreciated that other kinds of materials besides athletic tape could be used for wearable applications. For example, the optical fibers could be attached, sewn, or otherwise integrated into a flexible elastic compression bandage and used in the same manner as the athletic tape. And as further discussed herein, such optical fibers can be installed in wearable fabrics or other materials for use in sensing.

Stretch fiber optics made from silicone-clad polyurethane fibers work as low-noise, low hysteresis strain sensors that can be stitched to textiles for wearable applications. The Example with sample embodiments demonstrates a sensor able to recognize shape changes in muscles, detecting 2-3% changes in length corresponding to weight bearing activities. Weight bearing provides context for functional fitness tests, which include questions on whether a patient relies on furniture to take weight off joints or to provide balance. At least according the configurations used in the Example, textile-induced microbending required the embedded sensor to have a ˜10% pre-strain for a monotonic signal (FIG. 4D), while a non-embedded fiber had only monotonic behavior at positive strains (FIG. 2A).

Beyond adding analog strain sensitivity to a wearable, breathable material, sewable optical threads according to some embodiments may carry digital signals over lengths of 25 cm or more. In practice lengths of stretchable strain sensors may be shorter than 25 cm or many orders of magnitude longer than 25 cm. These “stretchy” high speed optical communication links may be preferable to users over wireless methods for securely transmitting biometric data over wearable sensor networks. In terms of high speed communication for remote monitoring using optical stretch fibers, in some embodiments light pulses are sent over a fiber to pass serial signals, if the amplified signal is high enough to count as a digital “1.” The absorption coefficient of the stretchable polymer determines the range of fiber lengths that work for sensing and communication in a given application.

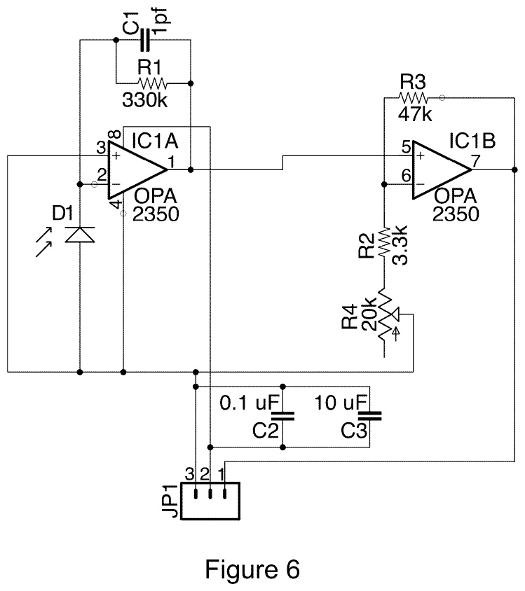

FIG. 6 provides an exemplary detection circuit 600 comprising a two-stage amplifier with a single-sided power supply, which can work with the voltage output available from standard microcontroller boards (2.7-5V).

### Example

Following is a description of sample embodiments used for prototypical testing. The following description may characterize some embodiments but should not be construed as necessarily limiting all embodiments. To parallel the description above, the same Figures may in some instances be used. It should be understood that in such cases the figures may be simultaneously illustrative of characteristics possessed by many embodiments as well as characteristics possessed by comparatively few embodiments.

FIGS. 1A and 1B show an optical fiber comprising a polyurethane core and a silicone cladding. In some embodiments, the core has a refractive index (n_{core}) of 1.52, and the refractive index of the silicone cladding (n_{cladding}) is 1.43. The refractive indexes were determined by immersing the materials in index-matching liquids and measuring the index of the closest-matching liquids using a laser refractometer at 633 nm.

In some embodiments, the polyurethane fiber core is commercially available Stretch Magic® (Pepperell Braiding Co., Pepperell, MA). In some embodiments, the fiber core has a diameter between about 500 to 800 microns and the cladding is about 100-200 microns thick. The tensile strength is ˜ 1 kg for the 500 micron diameter material. Methods for applying the claddings include applying a translucent silicone coating (ELASTOSIL M 4641 A/B, Wacker Chemie AG) around the core. The cladding protects the interface from dust and oils that cause light to scatter out. Other cladding materials include low refractive index (n<1.45) high transparency or semi-transparent urethane elastomers, acrylic elastomers, and polyethylene elastomers.

In fibers of the kind illustrated in FIG. 1A, light attenuation increases in a stretched polyurethane fiber, with green and shorter wavelengths from a white light source being filtered out through absorption. Other materials for the core fiber include higher refractive index (n at least 0.05 greater than the cladding), high transparency silicone elastomers, acrylic elastomers, and polyethylene elastomers.

FIG. 1B depicts unstrained and strained fiber diameter (left) and output light intensity at the opposite end from the LED (right). FIG. 2A graphs intensity loss vs strain for three different silicone-clad 0.5 mm diameter polyurethane fibers with a resting length of ˜21 cm; each trace contains data points from at least three successive relaxation cycles for an individual fiber. An Industrial Fiberoptics IF-D91 photodiode was used as a light sensor for the intensity vs strain measurements. Intensity-vs-strain was tested for 6 different fibers undergoing multiple cycles. The intensity vs strain curves were collected by clamping the light emitter end to a metal frame (Actobotics, ServoCity Inc) and the photodetector end to a moving stage controlled by a stepper motor. The fiber was stretched and relaxed repeatedly by a microcontroller board (Arduino) moving the stage between a pair of limit switches, while the amplified photodiode signal (0-5V) was converted to a digital value and collected.

For the results of these measurements shown in FIG. 2A, differences between the three fibers are attributed to slightly different initial fiber lengths (+2%), and variations at the LED and detector housings. The transmitted light intensity drops exponentially with length, but with attenuation that can be detected on the cm scale instead of the kilometer length scale as seen in comparable systems such as silica fiber optics. The key effect of stretching is increased attenuation due to a longer path in the absorbing material.

Strain sensing tests were carried out with a 935 nm infrared source (IF-E91A), while the absorption-vs-length tests in FIG. 2B used both the infrared nm source and a visible red 650 nm source (IF-E97) with the IF-D91 detector installed on a printed circuit board (as was the infrared source). The photodetector signal was amplified on the PCB by an op-amp (OPA2350) with a gain of 5 V/microamp. Typical photocurrents were in the single microamp range. In the strain measurement system used, the emitter (i.e., light emitter) and detector circuits were installed on a linear translation stage (Actobotics, Inc) controlled by a microcontroller board (Arduino). The LED remained stationary while the detector was pulled along the rails by a belt attached to a motor-driven gear.

When each fiber in FIG. 2A was stretched from about 21 cm at 0% strain to 43 cm at the highest strain, the average measured loss vs length change for these was 0.446±0.05 dB cm-1. By comparison, FIG. 2B shows that the loss per cm of length due to combined absorbance and scattering in an unstretched fiber is 0.45 dB cm-1 at the 935 nm wavelength.

Given that motion at the ends of the fiber where it couples to the emitter and detector could affect the signal and interfere with sensing, it is important to control motion at the fiber end. Imaging the fiber end during stretching shows that with the fittings used in these experiments, fiber motion is below the resolution of the microscope, contributing at most ˜0.2 dB to the loss when the fiber is stretched to 100% strain. The loss from fiber lengthening during 120% strain is ˜10 dB, indicating this source of error associated with coupling is very small, if not negligible (e.g., on the order of 50× less than that due to fiber stretching).

FIGS. 2C-2E show other optical and mechanical properties of the silicone-clad polyurethane fiber. White light transmitted through the polyurethane rubber core emerges as yellow, with approximately 20 times greater transmission intensity at 700 nm than 550 nm (FIG. 2C). The polyurethane rubber transmits well at wavelengths between 650-1000 nm and possibly beyond, except for a low-transmission notch between 870-930 nm. The material is compatible with the visible red (650 nm) and near IR (850 nm) wavelengths used for optimum transmission in PMMA plastic optic fiber. An 850 nm LED and TSL-12T integrated detector (AMS-TAOS USA Inc) or IF-D91 photodiode (Industrial Fiberoptics Inc), designed for conventional fibers, proved to be good matches for the polyurethane fiber in this work. A 935 nm IF-E91A LED also worked well with the fiber and the IF-D91 detector, and was used in the stretching experiments described herein. These sources are illustrated on the spectrum in FIG. 2C. Because the 935 nm source is near the absorption notch, it shows a slightly higher attenuation constant (0.45 dB cm-1) than the 650 nm source (0.37 dB cm-1) in FIG. 2B, where absorption was measured through an unstrained fiber that was cut to successively shorter lengths between measurements.

Additionally, FIG. 2D shows how the clad fiber diameter d reduces with stretching, as described in Equation (2):

$$\frac{d}{{d}_{0}} = {( 1 + \frac{Δ L}{{L}_{0}} )}^{- v} \qquad ( 2 )$$

where d₀ is the original, unstretched fiber diameter, L₀ is the original length, ΔL is the change in length, and v is the Poisson's ratio of the material. The clad/coated fiber comes close to having an ideal Poisson's ratio of 0.5 for an incompressible material; its diameter-vs-length behavior will determine how it interacts with overlying stitches when attached to a textile by overlying stitches.

The first time the fibers reach 100% strain, the light intensity is 20-25% lower than it is at 100% strain in subsequent cycles. These lasting changes could be caused by rupture of the weakest bonds and realignment of the polymer chains. When the fiber is returned to the start position, it can take minutes to become taut again. After the first cycle, the path is repeatable (FIG. 2A) and the complete stretch-and-relax cycle has a small amount of hysteresis (FIG. 2E). The hysteresis error is 4-5% strain over the 5-100% strain range.

In FIG. 2B, absorption coefficients are shown at two wavelengths: 650 nm, dashed line, and 935 nm, solid line, respectively. These measurements were made by measuring the transmitted light intensity through a fiber, then cutting off a few mm and measuring again. Some degree of noise is seen in the data for FIG. 2B and originates from having to re-align the cut fiber with the detector for each measurement. In FIG. 2C, normalized fiber transmission is shown for the visible and near infrared spectrum. In FIG. 2D, fiber diameter decreases with stretching, and FIG. 2E graphically shows results from three stretch-and-relax cycles with a roundtrip time of ˜1 minute.

In measuring the optical and mechanical properties of silicone-clad Stretch Magic® polyurethane fiber, each fiber was 0.5 mm diameter and coated with 0.25 mm thick coating of M4641 silicone. Various coating techniques exist or can easily be used for the coating step. For the studies described herein, the 0.5 mm diameter fibers was coated with silicone by pulling the through a syringe filled with mixed, uncured silicone and out through a dispensing Luer tip (Howard Electronics Inc.) with the desired diameter. For example, an 18 gauge tip was used for the first coat, and 16-gauge tip for a second coat applied after the first coat was cured. Because the silicone will bead up on the fiber in about 5-10 minutes, the silicone was cured by reeling the freshly coated fiber past a heat lamp (250 W infrared incandescent heat bulb, Sylvania). The fiber ran 3 cm from the face of the bulb, reaching an approximate temperature of 50 C and a take-up motor was set so that the fiber spent 5 minutes in the 50 C zone. This dwell time was long enough to cure the silicone cladding all around the fiber. The above technique can be adapted for larger scale use, or different methods and techniques can be used or adapted as known in the art.

FIG. 3A illustrates a system for coating a fiber such as but not limited to a polyurethane fiber with a cladding formed from a material such as but not limited to silicone. The coating setup consists of or comprises a reel run by a stepper motor to pull the urethane fiber up through a syringe filled with uncured silicone. Red or clear M4601 silicone is suitable, among other alternatives. In this exemplary setup, the fiber moved past a heating lamp with a dwell time of approximately 5 minutes between the 200 Watt infrared bulb and an empty aluminum reflector from another lamp. This dwell time was adjusted so it was long enough for the coating to cure, preventing it from forming drips or sticking to itself on the uptake reel. The overall length of the setup from reel to reel was ˜1 meter, with the infrared bulb at about ⅔ the way toward the uptake reel. Keeping the heater away from the syringe, and applying an ice pack to the syringe extended the working time of the silicone supply to ˜2 hours before it solidified.

FIGS. 4A to 4E illustrate couching and its effects on the signal transmitted through the fiber. Here couching uses a sewing machine foot to align a thick fiber inside a zig-zag stitch on a sewing machine. The fiber was couched to self-adhesive athletic tape (KT Tape, KT Health) using a Brother Innov-is 80 sewing machine with a couching foot (Brother SA157 or SA148 For thicker fibers) and a zig-zag stitch with 3 mm pitch and 2 mm width. A soluble backing (Sticky Fabri-Solvy, Sulky of America) covered the sticky side of the tape during sewing (the backing was removed in water) and small sewable PCBs were designed to hold a compact LED source and integrated detector (TSL-12T, AMS-TAOS USA Inc.). The TSL-12T was wrapped in black silicone tape to prevent ambient light from saturating the signal, and a 18 KΩ resistor was put in series with the LED to reduce its intensity to a level that worked with the 0-5V output range. Unlike the fiber-only experiments described above in connection with FIGS. 1A, 1B, and IC and FIGS. 2A-2E, the load was applied to the fiber via the textile instead of having these miniaturized sources and detector carry the mechanical load.

For testing using a setup as depicted in FIG. 4C, optical fibers were installed in athletic tape with zig zag stitch pitches of 3 mm and widths of 2 mm. Miniaturized circuits for wearable sensing over a fabric were constructed by applying small emitter and detector components to the edge of printed circuit boards in line with the optical fiber. A first circuit comprised an 850 nm infrared emitter in series with 150 ohm resistor having pins for connection to a standard circuit board. The lower circuit comprised an integrated photodetector TSL-12T (AMS-TAOS USA Inc). Fibers were strain relieved with monofilament braid (Cortland Line Company, Cortland NY) and secured to the circuit boards with RTV silicone (Loctite Inc).

For the testing of optical fibers installed in athletic tape, clamps were 3D printed to attach the athletic tape to the same linear translation stage used in the fiber pulling testing according to the system shown in FIG. 1C. The emitter and detector were positioned on left and right sides, respectively. The three optical fibers were installed in stretchable athletic tape. In this testing, strain was applied to the tape instead of the fiber housings, meaning the small emitter and detector circuits did not need to hold tightly to the fibers.

As the fiber stretched, microbending was reduced but the path length increased. These competing effects mean that transmitted light intensity increases with strain until approximately 10-15% strain, where it begins a monotonic dropoff. At higher amounts of strain (γ>25%), the overlying threads compress the fiber and the signal drops faster than the material's K˜0.4 dB cm⁻¹ absorption coefficient (FIG. 4D). The stretched fiber thins asymptotically (FIG. 2D) while the overlying threads flatten toward the textile surface, pinching the fiber.

For testing purposes, multiple fibers were couched to KT Tape. FIG. 4D graphs the transmitted signal through the couched fiber during stretching phases according to the relationship between fiber length and intensity gain. FIG. 4E plot shows five cycles of stretching and relaxing the KT tape with embedded fiber.

The KT tape with couched fibers was further tested in a wearable application. FIGS. 5A and 5B show how KT tape with couched fibers was used to detect changes in muscle thickness while worn above the knee as the wearer shifted weight from one leg to the other. The tape was strained before application to the skin; kinesiology tape is designed to be applied to the skin at between 0 and 50% strain. Pre-straining the tape ensured that the weight bearing and non-weight bearing lengths were in the monotonic “lengthening” regime of FIG. 4D. The original optical fiber length was 16.4 cm. The strained fiber went from 19 cm (15.8% strain) to 19.5 cm (18.9% strain) during the shift from non-weight bearing to weight bearing (FIG. 5A).

Because the tape's radius of curvature also changes during weight shifting, the effect of bending on the transmitted intensity was also measured. When the un-strained tape was wrapped around 4 different cylinders on the scale of small arm/leg joints (7-10 cm diameter), the signal remained at 3±0.05 V. The transmitted intensity is much more sensitive to bending at sub-cm bending diameters, and accordingly light may be seen escaping from the bent fiber. In this fashion, optical signals based on light passing through the fibers were obtained and measured with a photodetector. In use, the source of the light passing through the fiber can be ambient light from the environment that enters through an opening at a first end of the fiber and exits through an opening at a second end proximal to the detector.

FIG. 5B shows ten cycles of weight bearing (5 seconds) and non-weight bearing (5 seconds). The weight-bearing signal averaged 3.07 V, while the non-weight bearing averaged 3.45 V, for a stretching-induced loss of 0.5 dB for the 0.5 cm length change during weight shifting, giving a shift of 1 dB cm-1 on the wearable device. This shift is in the right direction, but is greater than the 0.26 dB cm-1 seen in the “lengthening” region of FIG. 4D, where the textile was not in contact with an underlying surface. The additional shift to compression of the fiber likely resulted from muscle expansion as it pushes into the threads.

Beyond adding analog strain sensitivity to a wearable, breathable material, the sewable optical threads carried digital signals over 25 cm lengths. The absorption coefficient of the stretchable polymer determines the range of fiber lengths that work for sensing and communication in a given application. For the Stretch Magic® polyurethane fiber described herein, there was an attenuation K ˜0.3-0.5 dB cm⁻¹ with near IR light.

It has been determined that stitching a zig zag stitch over the top of any such fiber will hold it in place during stretching of the underlying textile. An inexpensive couching machine foot will create uniform stitches without damaging the fiber and works with a basic sewing machine. In this configuration, it was determined that 500-800 micron core diameter, single-clad polyurethane optical fibers as described herein are able to transmit measurable amounts of light over sufficient distances (e.g., 25 cm) after sewing, with a higher quality signal likely coming from the 800 micron diameter polyurethane fiber, with further improvements in transmission being associated with a double coating of M 4641 silicone, for a combined diameter of 1.25 mm.

In terms of high speed communication for remote monitoring using optical stretch fibers, in some embodiments light pulses are sent over a fiber to pass serial signals, if the amplified signal is high enough to count as a digital “1.” The inventors successfully transmitted characters on an unstrained 25 cm long fiber by a microcontroller board (Arduino Mega) without errors at 115,200 bps using the 935 nm LED. To the extent rate was limited by the LED switching speed; changing to a 100 Mbps, 870 nm LED (IF-E91, Industrial Fiberoptics) enabled communication at the next standard rate of 230,400 bps.

It is to be understood that the embodiments described and/or claimed herein are not limited in their application to the details of the teachings and descriptions set forth herein, or as illustrated in an example. Rather, it will be understood that the embodiments are capable of being practiced or carried out in multiple ways, according to many alternatives based on these descriptions and teachings.

Further, it will be understood that the phraseology and terminology used herein is for the purpose of description and should not be regarded as limiting. The use herein of “including,” “comprising,” “e.g.,” “such as, for example,” “containing,” or “having” and variations of those words is meant in a non-limiting way to encompass the items listed thereafter, and equivalents of those, as well as additional items. Accordingly, the foregoing descriptions are meant to illustrate a number of embodiments and alternatives, rather than limiting to the precise forms and processes disclosed herein. The descriptions herein are not intended to be exhaustive. It will be understood by those having ordinary skill in the art that modifications and variations of these embodiments are reasonably possible in light of the above teachings and descriptions.

1.-69. (canceled)

70. A strain sensor, comprising a textile,
one or more optical fibers attached to the textile,
threads configured to prestrain the textile and one or more optical fibers by 15-25%,
wherein the optical fibers have an elastic response for strains of up to at least 100%.
